(19) United States
(12) Patent Application Publication
(10) Pub. No.: US 2022/0226080 A1
(43) Pub. Date: Jul. 21, 2022

(54) Dental Implant Having Different Degrees of Surface Roughness

(71) Applicant: Vita Zahnfabrik H. Rauter GmbH & Co. KG, Bad Säckingen (DE)

(72) Inventor: André Schöne, OT Stresow (DE)

(21) Appl. No.: 17/615,220

(22) PCT Filed: Feb. 12, 2020
(86) PCT No.: PCT/EP2020/053630

§ 371 Date: Nov. 30, 2021

(30) Foreign Application Priority Data
Jun. 4, 2019 (EP) 19178173.1

(51) Int. Cl. A61C 8/00

(52) U.S. Cl. CPC A61C 8/0037; A61C 8/0075; A61C 2008/0046; A61C 8/0025; A61C 8/0012

(57) ABSTRACT

The invention relates to a dental implant having various regions with different degrees of surface roughness.

The present invention relates to a dental implant having different regions with different degrees of surface roughness.

Implants are artificial materials implanted into the body that are to remain within the body permanently or at least for an extended period of time. Dental implants are inserted into the jaw, where they adopt the function of an artificial tooth root as supports of prosthetic care articles. For this purpose, they are either turned by means of a screw thread or inserted into the jaw. The aim is for the implant to connect with the surrounding bone to form a strong and extremely load-resistant support unit. The use of such dental implants has the advantage that the adjacent teeth need not be recurred to for attaching the prosthetic care articles, such as bridges or crowns, so that the adjacent teeth need not be ground.

The dental implants are expected, on the one hand, to offer a stable support for the artificial tooth mounted thereon, and on the other hand, to undergo a quick and permanent connection with the surrounding tissue. For example, to promote ingrowth into the dental implant, it has proven advantageous to provide the implant with a roughened surface that facilitates the adhesion of bone cells. In contrast, there is a demand for a surface on which the prosthetic care article can be attached and connected.

EP 0 388 576 describes an implant provided with a porous surface and intended for insertion into a bone, whose surface has a microroughness with a pore size on the order of 2 μm or less.

DE 10 2005 006 979 discloses an enossal dental implant comprising an implant body formed for insertion into a jaw, and an implant post having a connection zone for attaching a prosthetic structure, in which at least the implant body is made of a ceramic material, and in which the implant is formed in at least two parts, so that the implant body and implant post are separate parts.

DE19828018 A1 describes a metallic self-tapping screw implant for anchoring artificial teeth, which has a mirror-polished neck.

EP 1 982 671 discloses a dental implant having a surface made of a ceramic material that has a topography with a core roughness depth Sₖ of less than 1 μm, and a skewness Sₛₖ of less than 0.

Although the prior art provides a number of solutions for how to promote the ingrowth of bone into the implant, there is still a demand for an implant that is accepted not only by hard tissue cells, such as bone cells, but also by soft tissue cells, such as gingival cells. It has been found that soft tissue cells prefer other surface structures than those preferred by hard tissue cells. Therefore, in order to avoid that retraction of the gum and thus exposure of at least part of the implant occurs, especially at the contact sites between the implant and gum, a solution must be found that promotes adhesion of both cell types, hard tissue cells and soft tissue cells, to the surface of the implant.

It is therefore the object of the present invention to provide a dental implant that not only shows a good ingrowth of the bone, but also allows for adhesion of soft tissue cells, especially gingival cells.

This object is achieved by a dental implant that has different regions with different degrees of surface roughness.

Therefore, the present invention first relates to a dental implant consisting completely or partially of ceramics comprising an enossal region (1) for ingrowth by a jaw bone, an abutment (3) for receiving a prosthetic care article, and an implant neck (2) arranged between said enossal region (1) and said abutment (3) for adhesion of gingival cells, characterized in that said enossal region (1), said implant neck (2) and said abutment (3) have respectively different average degrees of surface roughness Rₐ as measured according to DIN EN ISO 4287, in which said enossal region (1) has an average surface roughness Rₐ(E) of from 1.2 to 2.0 μm;

said implant neck (2) has an average surface roughness Rₐ(H) of from 0.04 to 0.2μm; and

said abutment has an average surface roughness Rₐ(A) of from 0.25 to 0.7 μm.

Because of the different degrees of surface roughness of the enossal region, the implant neck and the abutment, an improved adhesion of the different tissue cells is achieved, wherein it is avoided, especially by a corresponding surface roughness of the implant neck, that there is exposure of the implant neck from retracting gum. Thus, the dental implant according to the invention shows an excellent ingrowth time, also with different kinds of tissue, and offers a stable composite with the prosthetic care article. The different degrees of surface roughness of the various regions of the dental implant according to the invention can significantly shorten the duration of treatment, and the implant can be loaded already a short time after being inserted into the jaw, whereby an improved patient care is achieved. Thus, for example, the ingrowth time of the prior art implants is usually 3 months for the lower jaw and 6 months for the upper jaw, while the times could be significantly reduced for the implants according to the invention. For the implants according to the invention, the ingrowth time is about 2 months for the lower jaw and about 4 months for the upper jaw.

In order to achieve optimum retention of the implant in the jaw, it has proven advantageous to form the enossal region of the implant, i.e., the part of the implant that is inserted into the jaw and serves for anchoring in the jaw, in the shape of a thread. Therefore, one embodiment of the present invention in which the enossal region of the dental implant has a thread is preferred. Preferably, said thread is a self-tapping thread. In another embodiment, the dental implant does not have a self-tapping thread. Preferably, the length of the thread is selected to achieve sufficient anchoring within the bone.

Within the scope of the present invention, it has been found that the ingrowth speed into the implant can further be improved if the surface of the enossal region has, in addition to the macroroughness as proposed in the prior art, a microroughness superimposed thereon. Although the roughening of the surface increases the surface that is available to the bone cells for adhesion, it has been found that usual roughened surfaces, for example, those roughened by sanding, have sharp edges, which are avoided by bone cells.

Within the scope of the present invention, a surface that has, in addition to the macroscopic surface roughness, a microroughness superimposed thereon could be created by rounding off these edges, which is reflected in some porosity of the surface. Surprisingly, it has been found that the adhesion of the bone cells can be significantly improved if the macrostructured surface of the enossal region further possesses microroughness. Thus, in clinical trials with human CAL72 osteosarcoma cells, a clearly increased adhesion of the cells could be observed already after 24 hours, as compared to conventional implants. Accordingly, an embodiment of the present invention is preferred in which the surface of the enossal region of the dental implant according to the invention has a microroughness superimposing the macrostructured surface. This microroughness is manifested, in particular, by a porosity of the macrostructured surface and rounded surface structures.

In a preferred embodiment, the surface of the enossal region of the dental implant according to the invention has a microstructure with an average pore size of 2 μm or less, preferably from 0.5 to 1 μm. The average pore size of the macrostructure is about at 4 to 8 μm.

A correspondingly structures surface can be obtained, for example, by treating the surface by sanding, followed by chemically etching the surface, and a final thermal treatment. Therefore, an embodiment is preferred in which the enossal region of the dental implant according to the invention is obtained by treating the corresponding surface by sanding, followed by chemically etching, and a final thermal treatment. In a preferred embodiment, the thermal treatment is performed at temperatures of from 900 to 1500° C., preferably from 1200 to 1400° C. It was found that said treatment of the surface by sanding can yield significantly improved degrees of surface roughness, for example, over those obtained by machine processing of the surface. Further, it has surprisingly been found that the subsequent treatments of the surface, such as chemical etching and thermal treatment, have no influence on the actual macrostructure of the surface, but merely allow for the desired microstructure. A suitable method for obtaining such a surface is described, for example, in WO2009/007338.

In a preferred embodiment of the dental implant according to the invention, the enossal region has a macroroughness with values of the average surface roughness Rₐ(E_{macro}) of from 1.2 to 2.0 μm, preferably from 1.2 to 1.8 μm, especially from 1.4 to 1.6 μm, as determined according to DIN EN ISO 4287. Surprisingly, it has been found that the ingrowth time into the implant could be significantly shortened if the enossal region has an average surface roughness within the claimed range.

In a preferred embodiment, the enossal region of the dental implant according to the invention has a microroughness with values of the average surface roughness Rₐ(E_{micro}) of from 0.1 to 0.7 μm, preferably from 0.2 to 0.5 μm. More preferably, the ratio of macroroughness to microroughness, expressed as Rₐ(E_{macro})/Rₐ(E_{micro}) of the enossal region is from 20:1 to 3:1, preferably from 10:1 to 1.5:1.

The macroroughness of the surface can be determined, for example, by means of a Hommel Wave system (Hommel Wave, VS-Schwenningen, Germany). The microstructure of such a surface can be analyzed, for example, by confocal laser scanning microscopy (LEXT 0LS4000, Olympus, Tokyo, Japan).

The dental implant according to the invention is designed to achieve an optimum anchoring within the bone. In order to avoid a loss of bone from pressure load, for example, when the implant is seated too deep in the bone, it has proven advantageous if the maximum diameter of the implant neck does not exceed the diameter of the enossal region of the implant. Therefore, an embodiment of the dental implant according to the invention is preferred in which the dental implant has a pin-shaped design, wherein the maximum diameter of the implant neck is equal to or smaller than the maximum diameter of the enossal region. In this way, a good vertical bone preservation can be ensured. In each case, the “maximum diameter” designates a straight line going through the central axis of the dental implant, which forms the largest possible distance between two intersection points with a line running around the dental implant in the corresponding region orthogonally to the central axis, which corresponds to the perimeter of the dental implant in this region. The central axis of the dental implant corresponds to the axis running through the center of the implant in the direction of its largest dimension.

The implant neck is usually the part (region) of a dental implant that comes into contact with the soft tissue, especially the gum, and to which gingival cells are to adhere to ensure optimum grip and a long service life of the implant. In order to promote adhesion of the gingival cells, it has proven advantageous if the implant neck has a significantly lower surface roughness as compared to the enossal region of the implant. An optimum and sustainable adhesion of the gingival cells to the implant neck could be achieved if the average surface roughness of the implant neck is not higher than 0.25 μm. Therefore, an embodiment of the present invention is preferred in which the implant neck of the dental implant according to the invention has an average surface roughness Rₐ(H) of from 0.04 to 0.2 μm, preferably from 0.06 to 0.16 μm, more preferably from 0.08 to 0.12 μm. In a particularly preferred embodiment, the implant neck has an average surface roughness Rₐ(H) of less than 0.10 μm. Specifically preferred, the average surface roughness Rₐ(H) is within a range of from 0.04 to 0.95 μm, for example, from 0.05 to 0.09 μm. The average surface roughness is measured according to DIN EN ISO 4287. Within the scope of the present invention, it has been found that degrees of surface roughness within the claimed range on the one hand promote the adhesion of gingival cells, but on the other hand are also able to prevent a later retraction of the gum at the site of intrusion of the implant into the jaw.

In a particularly preferred embodiment, the dental implant according to the invention has a ratio of the surface roughness values of the implant neck and of the enossal region (Rₐ(H)/Rₐ(E)) of from 1:5 to 1:50, preferably from 1:10 to 1:20.

To improve the stability of the dental implant according to the invention, it has proven advantageous if the implant has a taper between the enossal region and the implant neck, i.e., where the transition from the hard tissue to the soft tissue occurs. Therefore, an embodiment is preferred in which the implant neck has a conical section in which the diameter preferably becomes smaller from the side facing the abutment to the side facing the enossal region.

In a particularly preferred embodiment, the implant neck has a conical section and a circumferential recess around the implant neck orthogonally to the central axis of the dental implant, especially a horizontal circumferential groove. Surprisingly, it has been found that the inclusion of such a groove promotes the effect of platform switching, wherein a good vertical bone maintenance was observed in this zone from the smaller diameter as compared to the enossal region of the dental implant according to the invention. The horizontal circumferential groove around the implant neck preferably directly borders the enossal region of the implant. In a particularly preferred embodiment, the groove is provided between the enossal region and the conical section of the implant neck. In a particularly preferred embodiment, this groove has a width of from 0.1 to 0.4 mm, preferably from 0.15 to 0.3 mm. As another advantage, the groove in the dental implant according to the invention can be employed for marking the ideal screwing depth of the implant.

The conical section of the implant neck preferably has the same average surface roughness Rₐ as the implant neck. In a preferred embodiment, the conical section of the implant neck has a height of from 0.5 to 3 mm, preferably from 1.5 to 2.5 mm.

The dental implant according to the invention further has an abutment for receiving a prosthetic care article. Said prosthetic care article is, for example, a bridge or a crown. The design and surface of the abutment are designed for an optimum bonding of the implant to a prosthetic care article. Therefore, in a preferred embodiment, the abutment has one or more recesses in order to enable a secure bonding to the prosthetic care article. For example, such a recess may be used to attach the prosthetic care article by means of clamps or according to the tongue and groove principle.

In a particularly preferred embodiment, the abutment has a lamellar design. Such lamellae are preferably one or more horizontal microgrooves in the surface of the abutment. This particular design of the surface of the abutment substantially improves the final attachment of the prosthetic care article.

In a further preferred embodiment, the abutment of the dental implant according to the invention has an average surface roughness Rₐ(A) of from 0.25 to 0.7 μm, preferably from 0.3 to 0.6 μm, more preferably from 0.3 to 0.5 μm, specifically from 0.25 to below 0.5 μm, for example, from 0.25 to 0.44 μm, respectively determined according to DIN EN ISO 4287. In a preferred embodiment, the abutment has a height of from 2.5 to 5.5 mm, preferably from 3.5 to 5.0 mm. Surprisingly, it has been found that an angular correction in the front tooth region when the prosthetic care article is fitted in can be avoided with an abutment in the selected height.

In a preferred embodiment, the dental implant according to the invention has a ratio of the average surface roughness values of the implant neck and of the abutment, expressed as Rₐ(H)/Rₐ(A), of from 1:20 to 1:1.5, preferably from 1:10 to 1:3.5, each of the surface roughness values being determined according to DIN EN ISO 4287.

In a further preferred embodiment, the dental implant according to the invention has a ratio of the average surface roughness values of the abutment to the enossal region, expressed as Rₐ(A)/Rₐ(E), of from 1:8 to 1:1.5, preferably from 1:5 to 1:3.5, each of the surface roughness values being determined according to DIN EN ISO 4287.

The dental implant according to the invention is preferably formed integrally. No particular requirements are to be demanded from the ceramic material of the implant, as long as it is biocompatible and safe for health. The dental implant according to the invention is completely or partially made of ceramic. Oxide ceramics have proven to be particularly suitable materials. Therefore, an embodiment is preferred in which the dental implant according to the invention is completely or partially made of an oxide ceramic comprising one or more oxides of the metals selected from aluminum, zirconium, yttrium, cerium, hafnium, magnesium, and mixtures and combinations thereof. In a particularly preferred embodiment, the dental implant according to the invention consists of or comprises completely or partially stabilized zirconium oxide. In a specifically preferred embodiment, yttria-stabilized zirconium oxide is used as the material for producing the dental implant according to the invention, wherein the proportion of yttria-stabilized zirconium oxide is preferably at least 90% by weight, more preferably at least 95% by weight, and specifically at least 98% by weight, respectively based on the total weight of the dental implant. A subsequent isostatic pressing process improves the material quality and decreases the risk of failure of the implant.

The material of the dental implant according to the invention is preferably tooth-colored. In this way, the foundation for a high aesthetics of the prosthetic care article can be laid, and the implant is prevented from “shimmering through”.

Details and advantages of the present invention are illustrated further by means of the following drawings, which are by no means to be construed as limiting the concept of the invention.

The ceramic dental implants of the present invention show advantages over metallic implants not only in terms of their aesthetic appearance and strength, but also in terms of the adhesion of bone cells and the spreading of gingival fibroblasts.

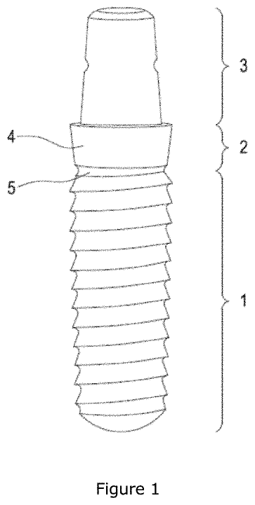

FIG. 1 shows, by way of example, a schematic representation of a ceramic dental implant according to the invention (based on an yttria-stabilized zirconium oxide) comprising an enossal region 1 (the part of the dental implant that serves for anchoring within the jaw), an abutment 3 and implant neck 2. There is further represented a conical section 4, which forms the implant neck 2, and a horizontal peripheral groove 5, which is provided below the conical section 4 and which is followed by the thread of the enossal region.

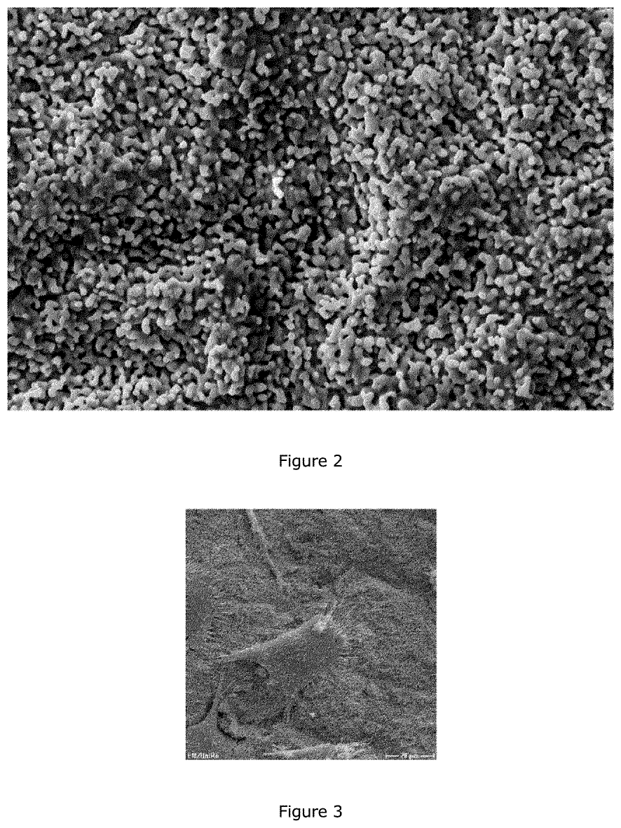

FIG. 2 shows a scanning electron micrograph of the surface of the enossal region of the dental implant according to the invention, wherein the microstructure of the macroscopically rough surface is clearly recognizable. The regions that appear darker in the photograph are located deeper and form a physical retention of the implant directly after the implant has been inserted into the bone. Without being bound by theory, it is assumed that the spongy bone displaced when the implant was inserted occupies this macroroughness and thus causes the improved primary strength of the implant according to the invention. The high acceptance of the bone cells observed is explained by the microroughness of the surface and creates the precondition of a gap-free ingrowth of the bone into the dental implant according to the invention.

FIG. 3 shows bone cells adhered to the surface of the enossal region of a dental implant according to the invention. The two-dimensional adhesion/expansion of the cells is clearly recognizable.

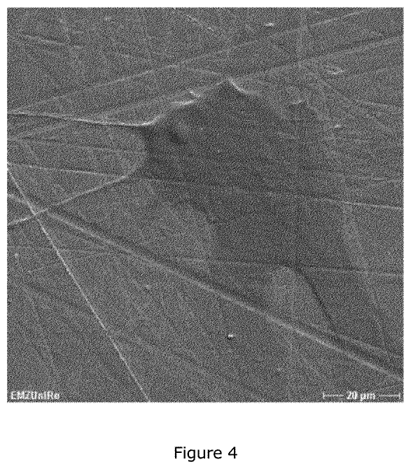

FIG. 4 shows the spreading of gingival fibroblasts after 24 hours on the implant neck of a dental implant according to the invention.

1. A dental implant consisting completely or partially of ceramics comprising
an enossal region (1) for ingrowth by a jaw bone,
an abutment (3) for receiving a prosthetic care article, and an implant neck (2) arranged between said enossal region (1) and said abutment (3) for adhesion of gingival cells, characterized in that said enossal region (1), said implant neck (2) and said abutment (3) have respectively different average degrees of surface roughness Rₐ as measured according to DIN EN ISO 4287, in which
said enossal region (1) has an average surface roughness Rₐ(E) of from 1.2 to 2.0 μm;
said implant neck (2) has an average surface roughness Rₐ(H) of from 0.04 to 0.2 μm; and
said abutment has an average surface roughness Rₐ(A) of from 0.25 to 0.7 μm.

2. The dental implant according to claim 1, characterized in that said enossal region (1) has a thread.

3. The dental implant according to claim 1, characterized in that said enossal region (1) has an average surface roughness Rₐ(E) of from 1.2 to 1.8 μm, especially from 1.4 to 1.6 μm, as measured according to DIN EN ISO 4287.

4. The dental implant according to claim 1, characterized in that said dental implant has a pin-shaped design, wherein the maximum diameter of the implant neck (2) is equal to or smaller than the maximum diameter of the enossal region (1).

5. The dental implant according to claim 1, characterized in that said implant neck (2) has an average surface roughness Rₐ(H) of from 0.06 to 0.16 μm, specifically from 0.08 to 0.12 μm, or up to 0.10 μm, as measured according to DIN EN ISO 4287.

6. The dental implant according to claim 1, characterized in that said implant neck (2) has a conical section (4) in which the diameter preferably becomes smaller from the side facing the abutment (3) to the side facing the enossal region (1).

7. The dental implant according to claim 1, characterized in that said implant neck (2) has a conical section (4) and a horizontal circumferential recess (5), especially a horizontal circumferential groove.

8. The dental implant according to claim 1, characterized in that said implant neck (2) has a horizontal circumferential groove (5) that directly borders the enossal region (1).

9. The dental implant according to claim 7, characterized in that said implant neck (2) has a horizontal circumferential groove (5) that is arranged between said enossal region (1) and said conical section (4) of the implant neck.

10. The dental implant according to claim 1, characterized in that said abutment (3) has one or more recesses.

11. The dental implant according to claim 1, characterized in that said abutment (3) has one or more microgrooves.

12. The dental implant according to claim 1, characterized in that said abutment (3) has an average surface roughness Rₐ(A) of from 0.3 to 0.6 μm, specifically from 0.3 to 0.5 μm, as measured according to DIN EN ISO 4287.

13. The dental implant according to claim 1, characterized in that said dental implant is integrally formed.

14. The dental implant according to claim 1, characterized in that said dental implant is completely or partially made of an oxide ceramic comprising one or more oxides of the metals selected from aluminum, zirconium, yttrium, cerium, hafnium, and magnesium.

15. The dental implant according to claim 1, characterized in that said ceramic is a glass ceramic.

16. The dental implant according to claim 1, characterized in that said enossal region has a microstructure with an average pore size of 2 μm or less, preferably from 0.5 μm to 1 μm.

17. The dental implant according to claim 1, characterized in that said abutment has a height of from 2.5 to 5.5 mm.

18. The dental implant according to claim 1, characterized in that the ratio of the average surface roughness values of the abutment to the enossal region, Rₐ(A)/Rₐ(E), is from 1:8 to 1:15, preferably from 1:5 to 1:3.5.
